(19) United States
(12) Patent Application Publication
(10) Pub. No.: US 2024/0001043 A1
(43) Pub. Date: Jan. 4, 2024

(54) MEDICATION DELIVERY DEVICE

(71) Applicant: BPREX PHARMA PACKAGING INDIA PVT. LTD., Peenya, Bengaluru Karnataka (IN)

(72) Inventors: Alwin JOGIN, Peenya, Bengaluru Karnataka (IN); Santosh ROTTI, Peenya, Bengaluru Karnataka (IN); Jasraj SINGH, Peenya, Bengaluru Karnataka (IN)

(21) Appl. No.: 18/257,942

(22) PCT Filed: Dec. 16, 2021
(86) PCT No.: PCT/EP2021/086297

§ 371 Date: Jun. 16, 2023

(30) Foreign Application Priority Data
Dec. 17, 2020 (GB) 2019965.9

(51) Int. Cl. A61M 5/32; A61M 5/20

(52) U.S. Cl. CPC A61M 5/326; A61M 5/2033

(57) ABSTRACT

A medication delivery device is provided and comprises a housing, a plunger within the said housing, a plunger hook, and a button, the button connects to a safety sleeve, disengaging safety sleeve from the plunger hook, thereafter the button engages with the plunger hook to push release the plunger held under spring pressure. The device further comprises a shield lock member to connect a pre-filled syringe (PFS) holder and a needle shield together. The shield lock member is activated after the dose is delivered.

The present invention relates to a medication delivery device, and particularly, although not exclusively, to a portable medication delivery device such as a pen injector.

An injector pen (also called a medication pen) is a device used for injecting medication under the skin.

The present invention provides a medication delivery device, like a pen injector for a simple, easy, safe and accurate dosing of drugs or biological products.

An aspect of the present invention provides a medication delivery device comprising a housing, a plunger within the said housing, a plunger hook, and a button, the button connects to a safety sleeve, disengaging safety sleeve from the plunger hook, thereafter the button engages with the plunger hook to push release the plunger held under spring pressure, the device further comprises a shield lock member to connect a pre-filled syringe (PFS) holder and a needle shield together, in which the shield lock member is activated after the dose is delivered.

An aspect of the present invention provides an auto-injector comprising of a housing, a plunger within the said housing, plunger hook, and a Button, Button connects to the safety sleeve, disengaging safety sleeve from the plunger hook, thereafter button engages with the plunger hook to push release the plunger held under spring pressure, the device further comprises of a locking element, as shield lock member to connect Pre Filled Syringe (PFS) holder and the needle shield together, shield lock member is activated only after the dose is delivered.

An aspect of the present invention provides a mono-dose pen injector device comprising a housing, a plunger within said housing, and a plunger hook, the device further comprises a locking member within the rear housing which comprises a plunger, a plunger hook and a safety sleeve, the device comprises a locking element comprising a shield lock member to hold a pre-filled syringe (PFS) holder and a needle shield together, the shield lock is activated only after a dose is delivered.

Some embodiments of the present invention provide or relate to a pen injector capable of delivering single dose of a liquid medicament contained therein without the need of priming the injector prior to administration and allow for administration of a dose of medicament in a simple, easy, safe and accurate dosing to patients by self-administration.

In some embodiments the plunger hook is provided with legs which hold the plunger by self strength.

A push/press button may be provided to cause release of the plunger.

The button may push plunger hook legs to release the plunger.

The plunger hook may be supported by a safety sleeve to be held in place, when not in use.

A shield hook may be hung onto the rear body.

The shield hook may be released when the plunger slides out of the mechanism,

The shield hook may latch onto the PFS holder and holds the needle shield in place thus linking the needle shield and PFS holder together after drug delivery, to avoid needle stick injury.

A plunger hook may move out of a safety sleeve.

Compressed spring force may release the plunger.

The plunger may move into the PFS and pushes a rubber bung to deliver medication.

A further aspect provides a pen injector device, which comprises: a housing, a plunger within said housing, a plunger hook, a locking member is provided within a rear housing, the locking member comprises the plunger, the plunger hook and a safety sleeve, a locking element is provided and comprise a shield lock to hold a PFS holder and a needle shield together, plunger hook legs hold the plunger by self strength, a button pushes the plunger hook legs to release the plunger, a spring guide sits within the plunger, a cap sleeve is provided, the plunger hook is supported by the safety sleeve to be held in place, when not in use, the shield lock is hung onto the rear housing, the shield lock is released when the plunger slides out of the mechanism, the shield lock latches onto the PFS holder and holds the needle shield in place thus linking the needle shield and PFS holder together after drug delivery, to avoid needle stick injury, the shield lock is activated only after the dose is delivered from the PFS in use.

The device may have four separate springs: i) a delivery spring pushes the plunger forwards; ii) a needle shield spring pushes the needle shield forwards; iii) a shield hook spring pushes the shield hook member forwards; and iv) a safety sleeve spring pushes the safety sleeve forwards.

In use the plunger may move inside the PFS and when this happens the shield lock moves along with the plunger and hooks into the PFS holder.

Once the shield lock hooks onto the PFS holder the needle shield may be locked down and it will not come by itself, and in which the needle shield pulls the shield lock and the PFS holder along with it i.e. they are interlocked.

The safety sleeve may slide out of the plunger hook and after this the button engages with the plunger hook and pushes the plunger hook legs; this releases the plunger, the plunger is already under delivery spring pressure whereby this pressure will push the plunger forward into the PFS.

The present invention may be provided as a medication delivery device.

Aspects of the present invention provide a pen injector device which comprises a housing, a plunger within said housing and plunger hook.

Embodiments may comprise a locking member within the rear housing which comprises plunger, plunger hook and safety sleeve.

Embodiments may comprise a locking element comprising a shield lock member to hold the PFS holder and needle shield together.

The shield lock may be activated only after the dose is delivered.

Aspects and embodiments of the present invention may comprise one or more of the following features:
1. Single dose/mono-dose device
2. Single use disposable device
3. Manual button actuated device.
4. Subcutaneous injection for solutions.
5. Used with PFS (Pre-Filled Syringe) of, for example, 1 ml volume
6. Maximum Delivered Dose=up to 1 ml (1 ml PFS)

Different aspects and embodiments of the invention may be used separately or together.

Further particular and preferred aspects of the present invention are set out in the accompanying independent and dependent claims. Features of the dependent claims may be combined with the features of the independent claims as appropriate, and in combination other than those explicitly set out in the claims. Each aspect can be carried out independently of the other aspects or in combination with one or more of the other aspects.

The present invention will now be more particularly described, by way of example, with reference to the accompanying drawings.

Example embodiments are shown and described in sufficient detail to enable those of ordinary skill in the art to embody and implement the systems and processes herein described. It is important to understand that embodiments can be provided in many alternate forms and should not be construed as limited to the examples set forth herein.

Accordingly, while embodiments can be modified in various ways and take on various alternative forms, specific embodiments thereof are shown in the drawings and described in detail below as examples. There is no intent to limit to the particular forms disclosed. On the contrary, all modifications, equivalents, and alternatives falling within the scope of the appended claims should be included. Elements of the example embodiments are consistently denoted by the same reference numerals throughout the drawings and detailed description where appropriate. The invention is not limited in the design and shape of the structure shown in the drawings.

The terminology used herein to describe embodiments is not intended to limit the scope. The articles “a,” “an,” and “the” are singular in that they have a single referent, however the use of the singular form in the present document should not preclude the presence of more than one referent. In other words, elements referred to in the singular can number one or more, unless the context clearly indicates otherwise. It will be further understood that the terms “comprises,” “comprising,” “includes,” and/or “including,” when used herein, specify the presence of stated features, items, steps, operations, elements, and/or components, but do not preclude the presence or addition of one or more other features, items, steps, operations, elements, components, and/or groups thereof.

Unless otherwise defined, all terms (including technical and scientific terms) used herein are to be interpreted as is customary in the art. It will be further understood that terms in common usage should also be interpreted as is customary in the relevant art and not in an idealized or overly formal sense unless expressly so defined herein.

The present invention provides a medication delivery device, like a pen injector for a simple, easy, safe and accurate dosing of drugs or biological products.

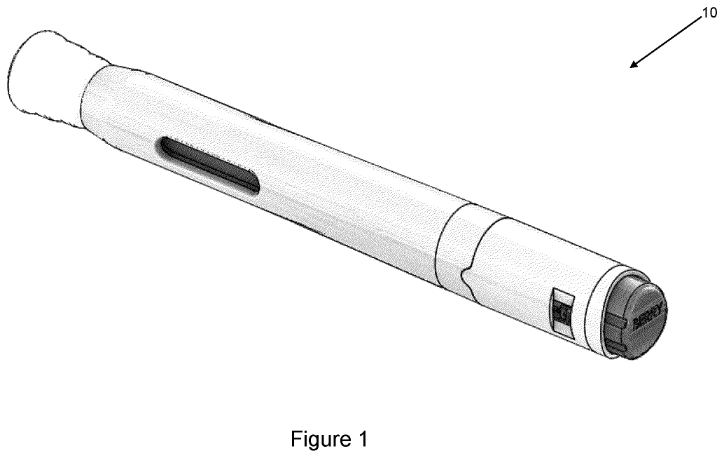

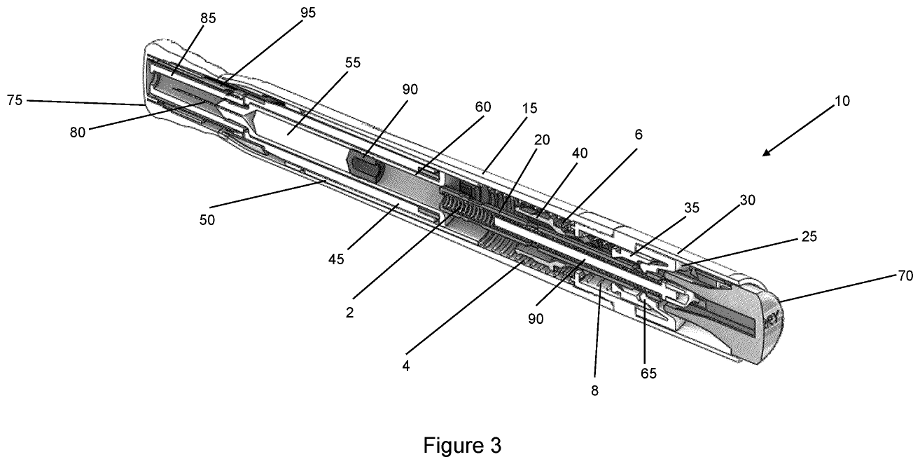

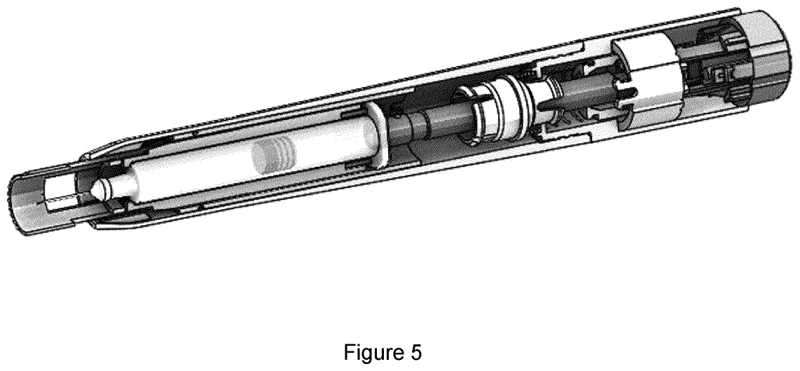

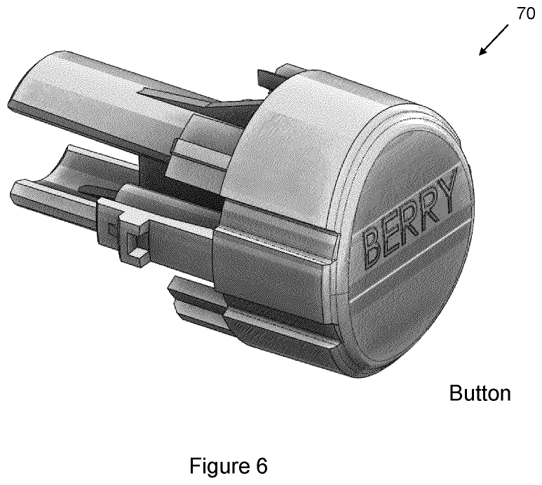

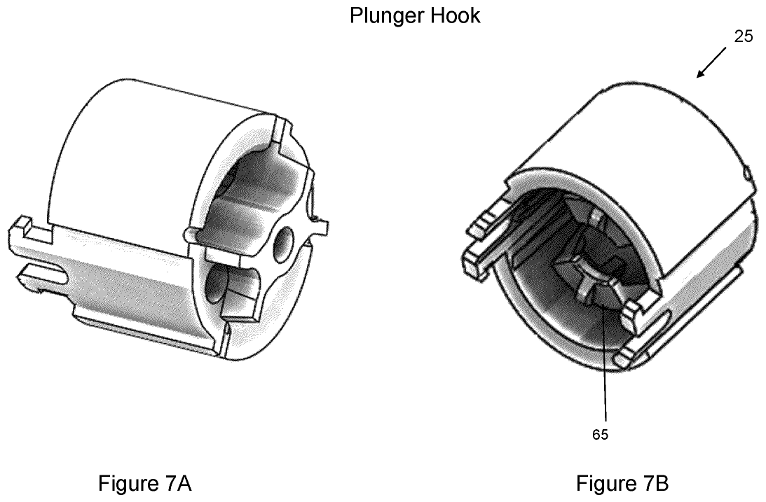

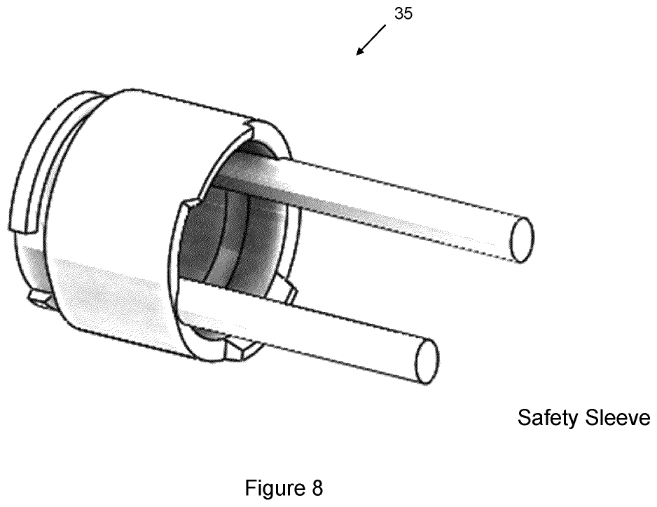

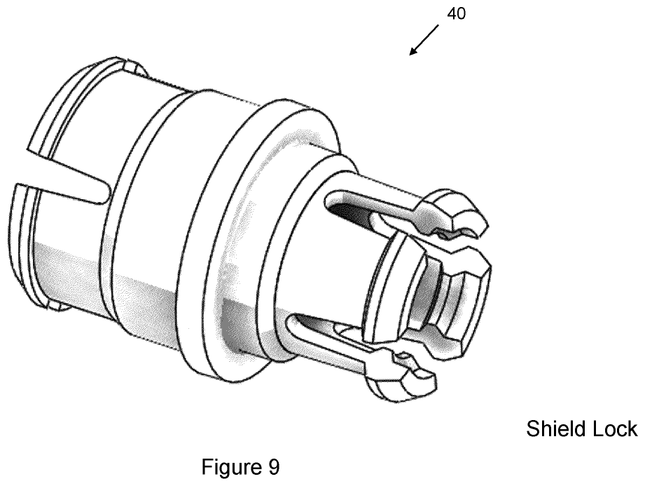

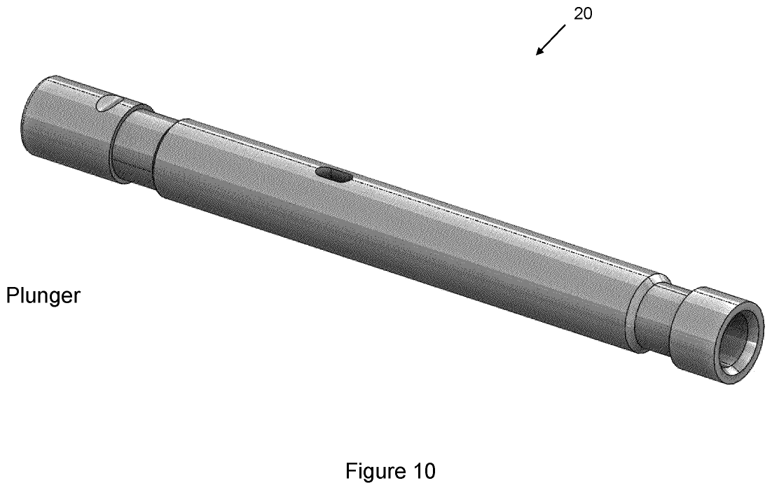

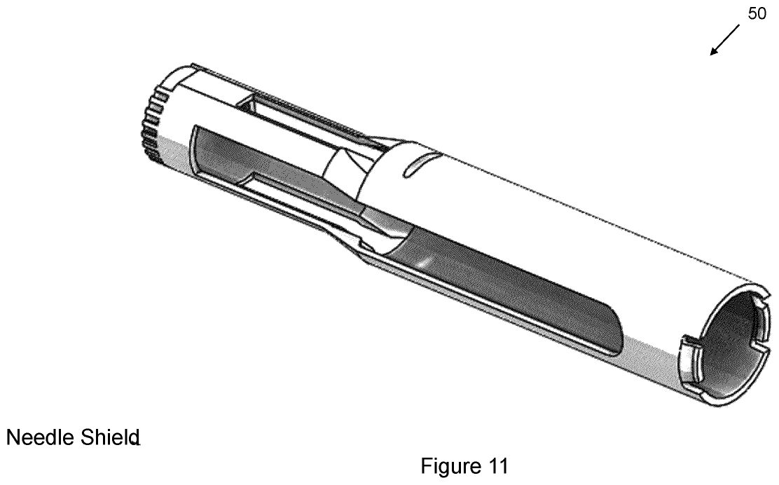

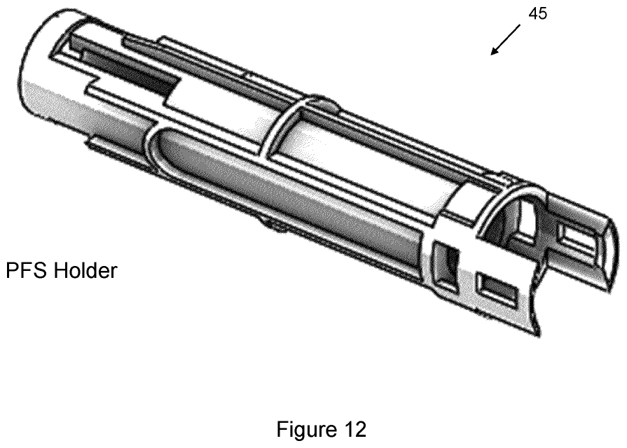

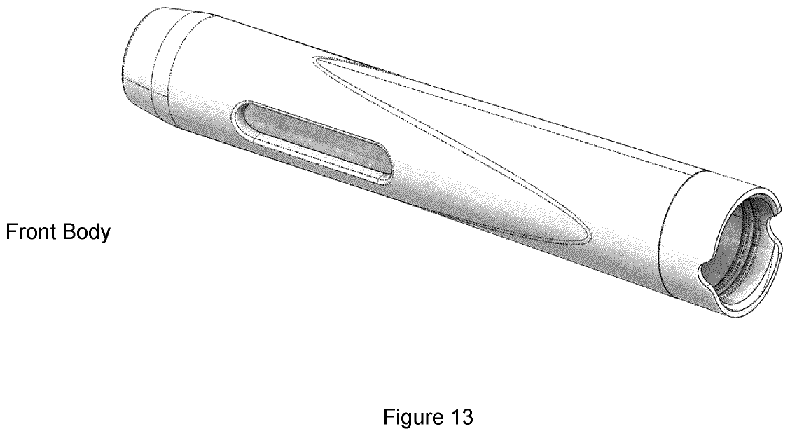

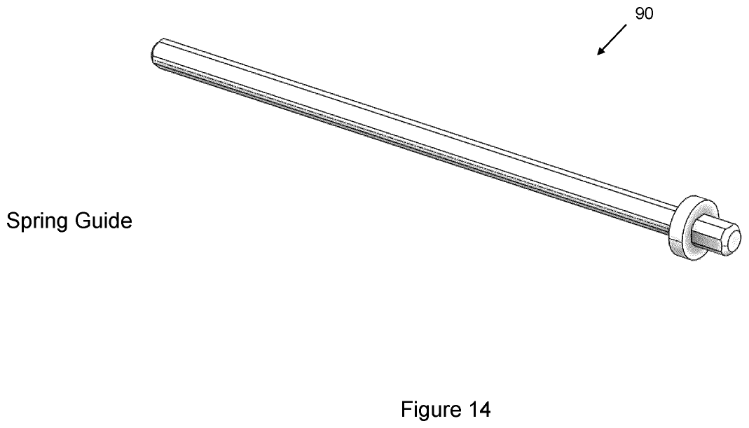

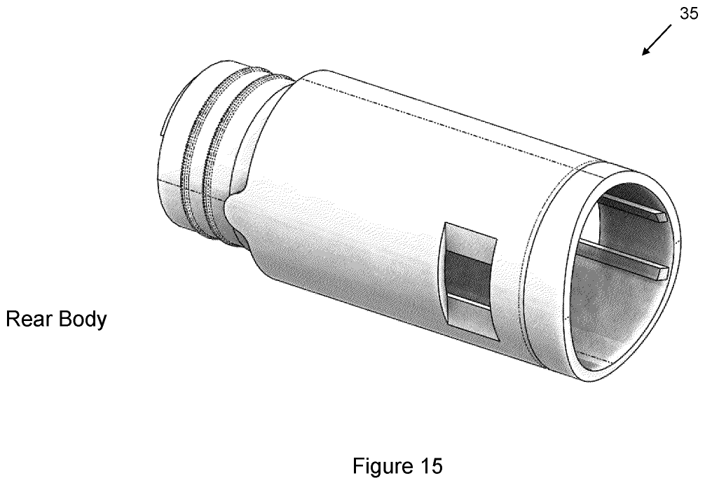

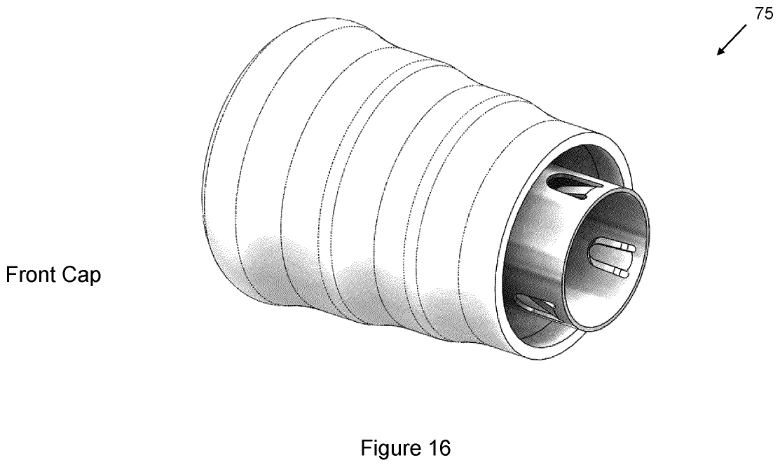

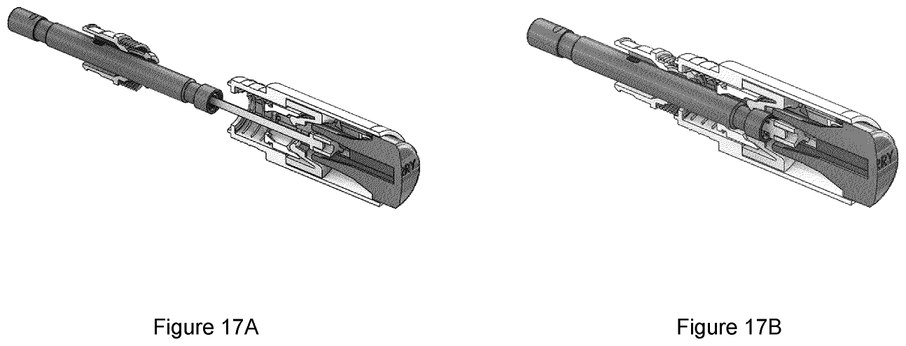

Referring first to FIGS. 1 to 17, an embodiment of the present invention provides a pen injector device 10, which comprises:
an elongate housing 15
a plunger 20 within said housing
plunger hook 25
a locking member within a rear housing 30 which comprises the plunger 20, the plunger hook 25 and a safety sleeve 35
a locking element comprising a shield lock 40 to hold a PFS holder 45 and needle shield 50 together; the shield lock is activated only after the dose 55 is delivered from the PFS 60 in use
plunger hook legs 65 hold the plunger by self strength
a button 70 pushes the plunger hook legs 65 to release the plunger 20
a spring guide 90 sits within the plunger 20
a cap sleeve 95
the plunger hook 25 is supported by the safety sleeve 35 to be held in place, when not in use
the shield lock 40 is hung onto the rear housing 35
the shield lock 40 is released when the plunger 20 slides out of the mechanism
the shield lock 40 latches onto the PFS holder 45 and holds the needle shield in place thus linking the needle shield 50 and PFS holder 45 together after drug delivery, to avoid needle stick injury

The device 10 has four separate (compression) springs:
a delivery spring 2 pushes the plunger 20 forwards
a needle shield spring 4 pushes the needle shield 50 forwards
a shield hook spring 6 pushes the shield hook member 40 forwards
a safety sleeve spring 8 pushes the safety sleeve 35 forwards

All of the springs are trying to push “forwards” i.e. away from the button 70.

In use the plunger 20 moves inside the PFS 60. When this happens the shield lock 40 moves along with the plunger 20 and hooks into the PFS holder.

Once the shield lock 40 hooks onto the PFS holder 45 the needle shield 50 is locked down and it will not come by itself. The needle shield 50 pulls the shield lock 40 and the PFS holder 45 along with it i.e. they are interlocked.

Safety sleeve 35, slides out of the plunger hook 25. After this, button 70 engages with the plunger hook 25 and pushes the plunger hook legs 65; this releases the plunger 20.

The plunger is already under delivery spring 02 pressure; this pressure will push the plunger 20 forward into the PFS.

The shield lock 40 provide a failsafe, activating only after dose delivery.

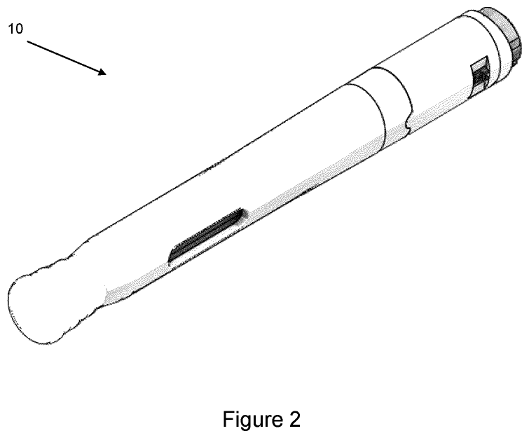

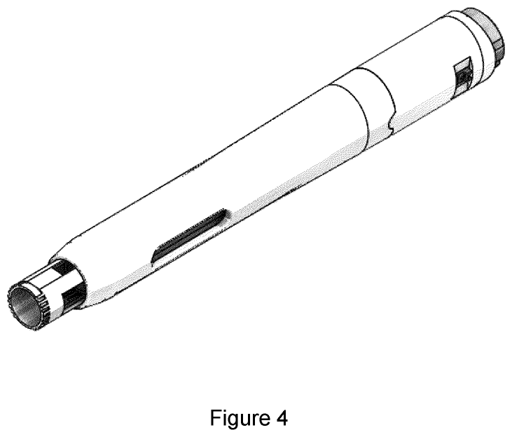

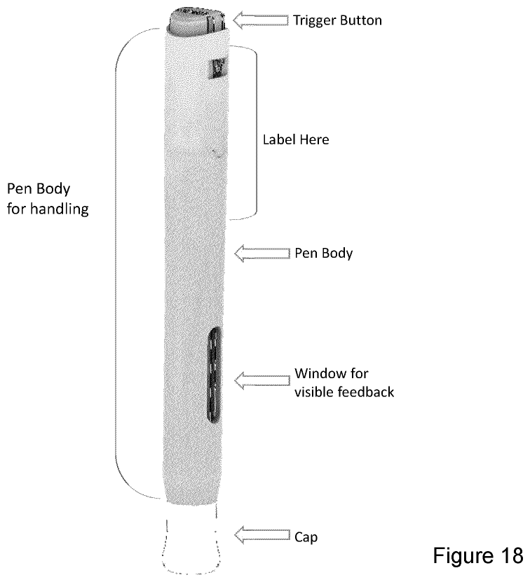

Referring now also to FIG. 18, operation of a device formed in accordance with the present invention is as follows:
1. Pick the pen 10 from primary packaging (see FIGS. 1 and 2).
2. Pull the front cap 75, out and away from the pen (see FIG. 4).
3. Observe the needle 80 is concealed inside the needle cap 85 at the cap end.
4. Observe the button 70 is projecting out of the body.
5. Sanitize the injection location and place the needle end on the injection area (skin) and press towards the skin.
6. Hold the pen firmly against the skin and press the button 70 on the other end.
i. Compressed spring force releases the plunger 20.
ii. Plunger 20 moves into the PFS 60 and pushes the rubber bung 90 to deliver the medication 55.
7. After the dwell time, the medicine window turns to purple colour (see FIG. 18), pull out the pen from the injection location.
8. Needle shield 50 moves out of the pen, concealing the needle inside 80.
9. Dispose the pen as instructed.

Although illustrative embodiments of the invention have been disclosed in detail herein, it is understood that the invention is not limited to the precise embodiments shown and that various changes and modifications can be effected therein by one skilled in the art without departing from the scope of the invention.

1-18. (canceled)

19. A medication delivery device comprising a housing, a plunger within the said housing, a plunger hook, and a button, the button connects to a safety sleeve, disengaging safety sleeve from the plunger hook, thereafter the button engages with the plunger hook to push release the plunger held under spring pressure, the device further comprises a shield lock member to connect a pre-filled syringe (PFS) holder and a needle shield together, in which the shield lock member is activated after the dose is delivered.

20. The medication delivery device of claim 19, wherein plunger hook legs are configured to hold the plunger by self-strength.

21. The medication delivery device of claim 19, wherein the button is configured to release the plunger.

22. The medication delivery device of claim 21, wherein the button is configured to push plunger hook legs to release the plunger.

23. The medication delivery device of claim 19, wherein the plunger hook is supported by a safety sleeve to be held in place, when not in use.

24. The medication delivery device of claim 19, wherein a shield hook is hung onto the rear body.

25. The medication delivery device of claim 24, wherein the shield hook is released when the plunger slides out of the mechanism.

26. The medication delivery device of claim 24, wherein the shield hook latches onto the PFS holder and holds the needle shield in place thus linking the needle shield and PFS holder together after drug delivery, to avoid needle stick injury.

27. The medication delivery device of claim 19, wherein a plunger holder is configured to move out of a trigger sleeve.

28. The medication delivery device of claim 19, wherein compressed spring force is configured to release the plunger.

29. The medication delivery device of claim 19, wherein the plunger is configured to move into the PFS and is configured to push a rubber bung to deliver medication.

30. An auto-injector comprising a housing, a plunger within the said housing, a plunger hook, and a button, wherein the button connects to a safety sleeve, wherein the safety sleeve is configured to disengage from the plunger hook, and thereafter the button is configured to engage with the plunger hook to push release the plunger held under spring pressure, wherein the auto-injector further comprises a locking element as shield lock member to connect a pre filled syringe (PFS) holder and the needle shield together, wherein the shield lock member is activated only after a dose is delivered.

31. A pen injector device, which comprises:
a housing,
a plunger within said housing,
a plunger hook,
a locking member provided within a rear housing, the locking member comprises the plunger, the plunger hook and a safety sleeve,
a locking element comprising a shield lock to hold a PFS holder and a needle shield together;
wherein plunger hook legs hold the plunger by self-strength,
wherein a button pushes the plunger hook legs to release the plunger,
wherein a spring guide sits within the plunger,
wherein a cap sleeve is provided,
wherein the plunger hook is supported by the safety sleeve to be held in place, when not in use,
wherein the shield lock is hung onto the rear housing,
wherein the shield lock is released when the plunger slides out of the mechanism,
wherein the shield lock latches onto the PFS holder and holds the needle shield in place thus linking the needle shield and PFS holder together after dose delivery, to avoid needle stick injury, and
wherein the shield lock is activated only after the dose is delivered from the PFS in use.

32. The device of claim 31, further comprising four separate springs.

33. The device of claim 31, further comprising four separate springs, including:
i) a delivery spring that pushes the plunger forwards;
ii) a needle shield spring that pushes the needle shield forwards;
iii) a shield hook spring that pushes the shield hook member forwards; and
iv) a safety sleeve spring that pushes the safety sleeve forwards.

34. The device of claim 31, wherein in use the plunger moves inside the PFS and when this happens the shield lock moves along with the plunger and hooks into the PFS holder.

35. The device of claim 31, wherein once the shield lock hooks onto the PFS holder the needle shield is locked down and it will not come by itself, and wherein the needle shield pulls the shield lock and the PFS holder along with it such that they are interlocked.

36. The device of claim 31, wherein the safety sleeve slides out of the plunger hook and after this the button engages with the plunger hook and pushes the plunger hook legs to release the plunger, and wherein the plunger is under delivery spring pressure whereby this pressure pushes the plunger forward into the PFS.
